(19) United States
(12) Patent Application Publication
(10) Pub. No.: US 2022/0172843 A1
(43) Pub. Date: Jun. 2, 2022

(54) LEARNING DEVICE, LEARNING METHOD, AND NON-TRANSITORY COMPUTER-READABLE MEDIUM

(71) Applicants: NEC CORPORATION, Tokyo (JP); Masao MIYASHITA, Obanazawa-shi, Yamagata (JP)

(72) Inventors: So YAMADA, Tokyo (JP); Riki ETO, Tokyo (JP); Junko WATANABE, Tokyo (JP); Masao MIYASHITA, Yamagata (JP); Marina GOTO, Yamagata (JP)

(73) Assignees: NEC CORPORATION, Tokyo (JP); Masao MIYASHITA, Obanazawa-shi, Yamagata (JP)

(21) Appl. No.: 17/601,857

(22) PCT Filed: Apr. 3, 2020
(86) PCT No.: PCT/JP2020/015290

§ 371 Date: Oct. 6, 2021

(30) Foreign Application Priority Data
Apr. 9, 2019 (JP) 2019-074032

(51) Int. Cl. G16H 50/20; G06K 9/62

(52) U.S. Cl. CPC G16H 50/20; G06K 9/6256

(57) ABSTRACT

A selection unit (11) in a learning device (10) inputs a plurality of “learning candidate data units.” The plurality of learning candidate data units are respectively related to a plurality of subjects including a plurality of cancer patients and a plurality of non-cancer patients. Further, each learning candidate data unit at least includes a “urine odor data unit” and a “cancer label.” Then, from the plurality of input learning candidate data units, the selection unit (11) selects part of the plurality of learning candidate data units as a “learning target data set,” based on a “selection rule.” By using the learning target data set selected by the selection unit (11), a determination model formation unit (12) forms a “determination model” for determining which of urine of a cancer patient and urine of a non-cancer patient a determination target urine odor data unit is related to.

### TECHNICAL FIELD

The present disclosure relates to a learning device, a learning method, and a control program.

### BACKGROUND ART

Technologies for detecting odor from a urine sample of a subject and generating a determination model for determining a disease from the detected odor (that is, sensing result data) have been proposed (such as Patent Literature 1).

### CITATION LIST

### Patent Literature

Patent Literature 1: Published Japanese Translation of PCT International Publication for Patent Application, No. 2004-531718

### SUMMARY OF INVENTION

### Technical Problem

However, the technology disclosed in Patent Literature 1 assumes every piece of sensing result data as data used for generation of a determination model (that is, learning target data) without making a selection, and therefore precision of the determination model may not reach a desired level.

An object of the present disclosure is to provide a learning device, a learning method, and a control program that can achieve improved precision of a determination model.

### Solution to Problem

A learning device according to a first aspect includes:

a selection unit configured to, from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, select part of the plurality of learning candidate data units as a learning target data set, based on a selection rule; and

a determination model formation unit configured to form a determination model for determining which of urine of a cancer patient and urine of a non-cancer patient a determination target urine odor data unit is related to, by using the selected learning target data set.

A learning method according to a second aspect includes:

from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, selecting part of the plurality of learning candidate data units as a learning target data set, based on a selection rule; and

forming a determination model for determining which of urine of a cancer patient and urine of a non-cancer patient a determination target urine odor data unit is related to, by using the selected learning target data set.

A control program according to a third aspect causes a learning device to execute processing of:

from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, selecting part of the plurality of learning candidate data units as a learning target data set, based on a selection rule; and

forming a determination model for determining which of urine of a cancer patient and urine of a non-cancer patient a determination target urine odor data unit is related to, by using the selected learning target data set.

### Advantageous Effects of Invention

The present disclosure enables provision of a learning device, a learning method, and a control program that can achieve improved precision of a determination model.

### BRIEF DESCRIPTION OF DRAWINGS

FIG. 1 is a block diagram illustrating an example of a learning device according to a first example embodiment.

FIG. 2 is a diagram for illustrating an example of a selection rule according to a second example embodiment.

FIG. 3 is a diagram for illustrating an example of a selection rule according to a third example embodiment.

FIG. 4 is a diagram for illustrating another example of a selection rule according to the third example embodiment.

FIG. 5 is a block diagram illustrating an example of a learning device according to a fourth example embodiment.

FIG. 6 is a block diagram illustrating an example of a cancer diagnostic system according to a fifth example embodiment.

FIG. 7 is a diagram illustrating an example of a collected data table according to the fifth example embodiment.

FIG. 8 is a block diagram illustrating an example of a learning device according to a sixth example embodiment.

FIG. 9 is a diagram for illustrating an example of a formation method of a learning target data set according to the sixth example embodiment.

FIG. 10 is a block diagram illustrating an example of a learning device according to a ninth example embodiment.

FIG. 11 is a block diagram illustrating an example of a cancer diagnostic system according to a tenth example embodiment.

FIG. 12 is a diagram illustrating a hardware configuration example of a learning device.

### DESCRIPTION OF EMBODIMENT

Example embodiments will be described below referring to drawings. The same or equivalent components are given the same sign in the example embodiments, and redundant description thereof is omitted.

### First Example Embodiment

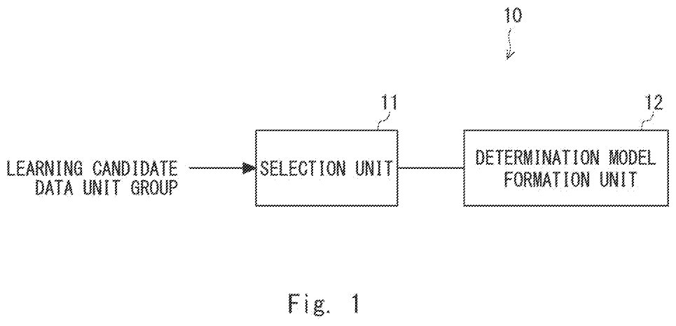

FIG. 1 is a block diagram illustrating an example of a learning device according to a first example embodiment. The learning device 10 illustrated in FIG. 1 is a device for learning a “determination model” for determining which of urine of a cancer patient and urine of a non-cancer patient a urine odor data unit of a determination target (hereinafter referred to as a “determination target urine odor data unit”) is related to. In FIG. 1, the learning device 10 includes a selection unit 11 and a determination model formation unit 12.

The selection unit 11 receives (inputs) a plurality of “learning candidate data units” (that is, a learning candidate data unit group). The plurality of learning candidate data units are respectively related to a plurality of subjects including a plurality of cancer patients and a plurality of non-cancer patients. Further, each learning candidate data unit includes at least a “urine odor data unit” and a “cancer label.” A urine odor data unit included in a learning candidate data unit is data related to odor detected from urine of a related subject, and, for example, a form thereof may be a vector including feature values of odor or a second-rank or higher tensor. The “cancer label” is a label at least indicating whether a related subject is a cancer patient or a non-cancer patient and, for example, may include a sub-label indicating whether the related subject is a cancer patient or a non-cancer patient. Specifically, for example, in addition to a sub-label indicating whether a related subject is a cancer patient or a non-cancer patient, the “cancer label” may include a sub-label indicating a type of cancer or a sub-label indicating progress of cancer.

Then, from the plurality of input learning candidate data units, the selection unit 11 selects part of the plurality of learning candidate data units as a “learning target data set,” based on a “selection rule.”

The determination model formation unit 12 forms the aforementioned “determination model” by using a learning target data set selected by the selection unit 11. The thus formed determination model is used in determination processing for determining which of urine of a cancer patient and urine of a non-cancer patient a determination target urine odor data unit a subject related to which is not determined to be a cancer patient or a non-cancer patient is related to. A learning method for forming the “determination model” is not particularly limited and, for example, may be logistic regression (LR), a support vector machine (SVM), a random forest (RF), or a neural network (NN).

As described above, from the aforementioned plurality of learning candidate data units, the selection unit 11 in the learning device 10 selects part of the plurality of learning candidate data units as a “learning target data set,” based on a “selection rule,” according to the first example embodiment. The determination model formation unit 12 forms the aforementioned “determination model” by using the learning target data set selected by the selection unit 11.

With the configuration of the learning device 10, a learning candidate data unit to be an actual learning target can be selected, and therefore improved precision of a determination model can be achieved.

### Second Example Embodiment

A second example embodiment relates to a specific example of the aforementioned “selection rule.” A basic configuration of a learning device according to the second example embodiment is the same as that of the learning device 10 according to the first example embodiment and therefore will be described with reference to FIG. 1.

A selection unit 11 in a learning device 10 according to the second example embodiment selects, from a plurality of input learning candidate data units, part of the plurality of learning candidate data units as a “learning target data set,” based on a “selection rule,” similarly to the first example embodiment.

The “selection rule” according to the second example embodiment includes a sub-rule (may be hereinafter referred to as a “first sub-rule”) for balancing, in a “learning target data set,” the number of learning candidate data units having a cancer label indicating a cancer patient with the number of learning candidate data units having a cancer label indicating a non-cancer patient.

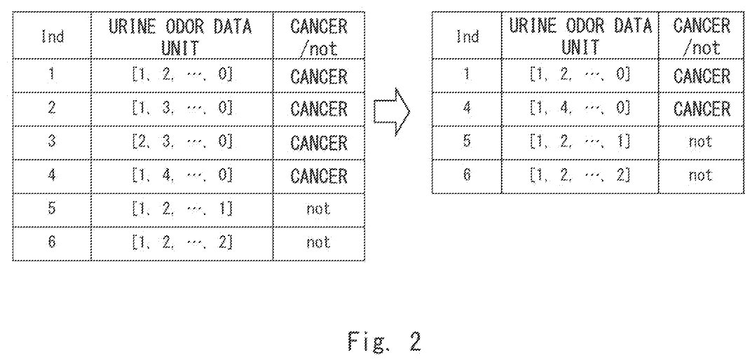

FIG. 2 is a diagram for illustrating an example of the selection rule according to the second example embodiment. A left-hand diagram in FIG. 2 illustrates an example of a learning candidate data unit group input to the selection unit 11, and a right-hand diagram in FIG. 2 illustrates an example of a “learning target data set” selected by the selection unit 11.

Each entry in the left-hand diagram in FIG. 2 is related to a learning candidate data unit and includes an index (Ind), a urine odor data unit, and a cancer label (CANCER/not) as items. Then, in the example in FIG. 2, entries 1, 4, 5, and 6 are chosen by the selection unit 11 as a learning target data set in accordance with the aforementioned first sub-rule, and entries 2 and 3 are excluded from the learning target data set. Two entries chosen as a learning target data set from the entries 1 to 4 having a cancer label indicating that a subject is a cancer patient may be randomly chosen or may be chosen based on a predetermined rule.

As described above, the selection unit 11 in the learning device 10 selects, from a plurality of input learning candidate data units, part of the plurality of learning candidate data units as a “learning target data set,” based on the selection rule, according to the second example embodiment. The “selection rule” includes a sub-rule for balancing, in the “learning target data set,” the number of learning candidate data units having a cancer label indicating a cancer patient with the number of learning candidate data units having a cancer label indicating a non-cancer patient.

With the configuration of the learning device 10, the number of learning candidate data units having a cancer label indicating a cancer patient can be balanced in a “learning target data set” with the number of learning candidate data units having a cancer label indicating a non-cancer patient. Thus, improved precision of a determination model can be achieved.

### Third Example Embodiment

A third example embodiment relates to a variation of the aforementioned “selection rule.” A basic configuration of a learning device according to the third example embodiment is the same as that of the learning device 10 according to the first example embodiment and therefore will be described with reference to FIG. 1.

Each learning candidate data unit according to the third example embodiment includes a “characteristic parameter” related to a subject in addition to the aforementioned “urine odor data unit” and the aforementioned “cancer label.” The “characteristic parameter” may take N (where N is a natural number equal to or greater than 2) pieces of k-th values (where k=1, . . . , N). In other words, the “characteristic parameter” may take at least a first value and a second value. For example, the “characteristic parameter” may be any one item out of “sex,” a “height,” a “weight,” a “comorbidity other than cancer,” and a “medication type” about a subject, or any combination of the above items.

A selection unit 11 in a learning device 10 according to the third example embodiment selects, from a plurality of input learning candidate data units, part of the plurality of learning candidate data units as a “learning target data set,” based on a “selection rule,” similarly to the first example embodiment and the second example embodiment.

The “selection rule” according to the third example embodiment includes a sub-rule (may be hereinafter referred to as a “second sub-rule”) for balancing, in a “learning target data set,” the numbers of learning candidate data units having k-th values. Specifically, the second sub-rule is a rule for balancing, in a learning target data set, the number of learning candidate data units having the aforementioned first value with the number of learning candidate data units having the aforementioned second value. The second sub-rule may be used with the aforementioned first sub-rule or may be used singly.

### Sub-Rule Example 1

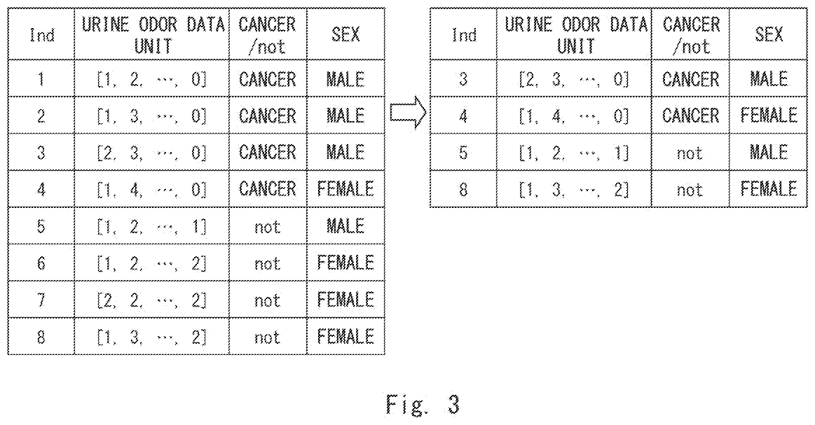

FIG. 3 is a diagram for illustrating an example of a selection rule according to the third example embodiment. A left-hand diagram in FIG. 3 illustrates an example of a learning candidate data unit group input to the selection unit 11, and a right-hand diagram in FIG. 3 illustrates an example of a “learning target data set” selected by the selection unit 11.

Each entry in the left-hand diagram in FIG. 3 is related to a learning candidate data unit and includes an index (Ind), a urine odor data unit, a cancer label (CANCER/not), and sex as items. In other words, sex is used as the aforementioned characteristic parameter in the example in FIG. 3. Then, entries 3, 4, 5, and 8 are chosen by the selection unit 11 as a learning target data set, and entries 1, 2, 6, and 7 are excluded from the learning target data set, in accordance with the aforementioned first sub-rule and the aforementioned second sub-rule, in the example in FIG. 3. An entry chosen as the learning target data set from among the entries 1 to 3 having a cancer label indicating that a subject is a cancer patient and having male as sex may be randomly chosen or may be chosen based on a predetermined rule. An entry chosen as the learning target data set from among the entries 6 to 8 having a cancer label indicating that a subject is a non-cancer patient and having female as sex may be randomly chosen or may be chosen based on a predetermined rule.

### Sub-Rule Example 2

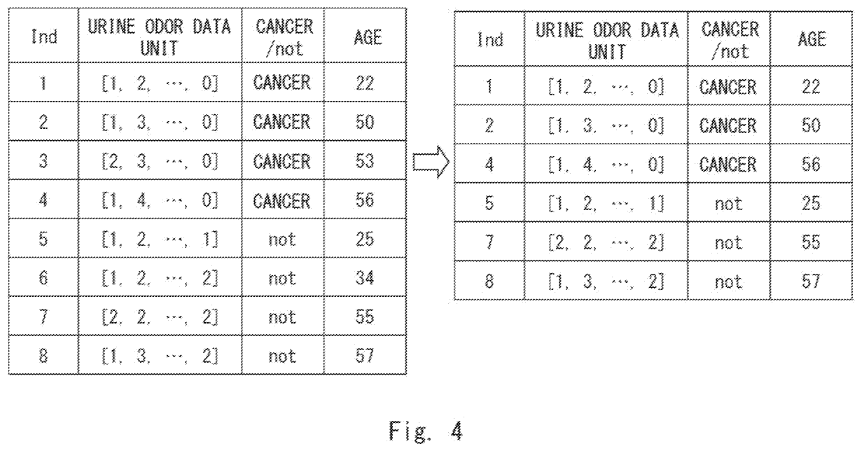

FIG. 4 is a diagram for illustrating another example of a selection rule according to the third example embodiment. A left-hand diagram in FIG. 4 illustrates an example of a learning candidate data unit group input to the selection unit 11, and a right-hand diagram in FIG. 4 illustrates an example of a “learning target data set” selected by the selection unit 11.

Each entry in a left-hand diagram in FIG. 4 relates to a learning candidate data unit and includes an index (Ind), a urine odor data unit, a cancer label (CANCER/not), and age as items. In other words, age is used as the aforementioned characteristic parameter in the example in FIG. 4. In a case of a characteristic parameter taking continuous values, such as age, a plurality of ranges related to a value of the characteristic parameter is defined, and the aforementioned second sub-rule may be a rule for balancing, in a “learning target data set,” the numbers of learning candidate data units in the ranges. For example, the aforementioned plurality of ranges include under 10, teens, twenties, thirties, forties, and so forth. Entries 1, 2, 4, 5, 7, and 8 are chosen by the selection unit 11 as a learning target data set, and entries 3 and 6 are excluded from the learning target data set, in accordance with the aforementioned first sub-rule and the aforementioned second sub-rule, in the example in FIG. 4.

### Sub-Rule Example 3

Further, a medication type given to a subject for treatment of a comorbidity other than cancer may be used as the aforementioned characteristic parameter. In this case, a “selection rule” may include a sub-rule for balancing, in a learning target data set, the number of learning candidate data units having a medication type indicating medication affecting urine of a subject and a cancer label indicating a cancer patient with the number of learning candidate data units having a medication type indicating medication affecting urine of a subject and a cancer label indicating a non-cancer patient. By using a learning target data set selected in accordance with the sub-rule in learning of a determination model, a bad effect of a determination model formed by a determination model formation unit 12 becoming a “determination model determining a medication type affecting urine of a subject” can be prevented.

Then, the determination model formation unit 12 according to the third example embodiment forms the aforementioned “determination model” by using the “learning target data set” selected by the selection unit 11, similarly to the first example embodiment and the second example embodiment. The determination model formation unit 12 may form a determination model by using a urine odor data unit and a cancer label as learning parameters used in learning of a determination model without using, in the learning, a characteristic parameter included in each learning candidate data unit in the learning target data set. The determination model formation unit 12 may instead form a determination model by using all of a characteristic parameter, a urine odor data unit, and a cancer label that are included in each learning candidate data unit in a learning target data set as learning parameters used in learning of a determination model.

As described above, the selection unit 11 in the learning device 10 selects, from a plurality of input learning candidate data units, part of the plurality of learning candidate data units as a “learning target data set,” based on a “selection rule,” according to the third example embodiment. Each learning candidate data unit further includes a “characteristic parameter” that is related to a subject and may take at least a first value and a second value. The “selection rule” includes a sub-rule for balancing, in a learning target data set, the number of learning candidate data units having the first value with the number of learning candidate data units having the second value.

With the configuration of the learning device 10, the numbers of learning candidate data units between characteristic parameter values can be balanced in a learning target data set. Thus, improved precision of a determination model can be achieved.

### Fourth Example Embodiment

A fourth example embodiment relates to a learning device that can accept specification of a sub-rule to be used out of a plurality of sub-rules different from one another included in a selection rule.

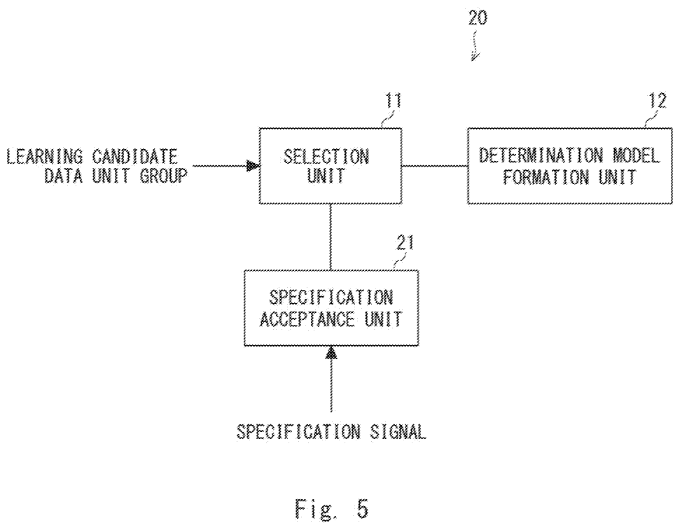

FIG. 5 is a block diagram illustrating an example of a learning device according to the fourth example embodiment. The learning device 20 in FIG. 5 includes a selection unit 11, a determination model formation unit 12, and a specification acceptance unit 21.

A “selection rule” according to the fourth example embodiment includes a plurality of sub-rules different from one another. The specification acceptance unit 21 accepts a “specification signal” indicating a single sub-rule or a combination of a plurality of sub-rules specified by a user operating an operation unit (unillustrated). Then, the specification acceptance unit 21 sets the single sub-rule or the combination of a plurality of sub-rules indicated by the specification signal to the selection unit 11 as a “selection rule to be used.” Thus, the selection unit 11 selects, from a plurality of input learning candidate data units, part of the plurality of learning candidate data units as a “learning target data set,” based on the “selection rule to be used” set by the specification acceptance unit 21.

As described above, the specification acceptance unit 21 in the learning device 20 accepts a “specification signal” indicating a single sub-rule or a combination of a plurality of sub-rules specified by a user operating the operation unit (unillustrated), according to the fourth example embodiment. Then, the specification acceptance unit 21 sets the single sub-rule or the combination of a plurality of sub-rules indicated by the specification signal to the selection unit 11 as a “selection rule to be used.”

With the configuration of the learning device 20, a “learning target data set” can be selected by using a selection rule matching user needs.

### Fifth Example Embodiment

A fifth example embodiment relates to a cancer examination system including a learning device.

### Outline of Cancer Examination System

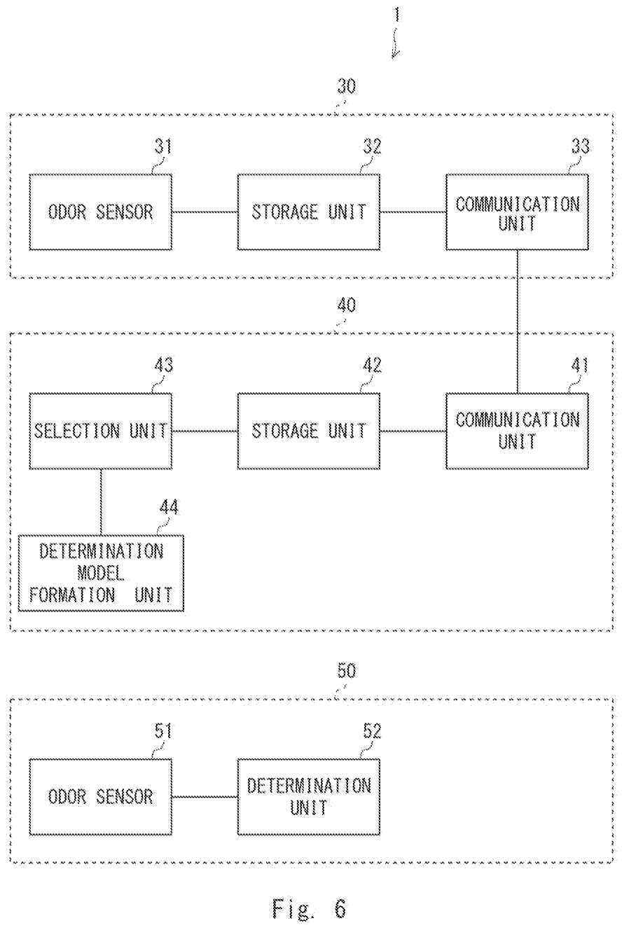

FIG. 6 is a block diagram illustrating an example of a cancer diagnostic system according to the fifth example embodiment. The cancer diagnostic system 1 in FIG. 6 includes a data acquisition device 30, a learning device 40, and a determination device 50. For example, the data acquisition device 30 may be installed in a hospital or a research institution. For example, the learning device 40 may be installed in a hospital or a research institution or may be constructed on a cloud. The determination device 50 may be installed in a determination institute determining which of urine of a cancer patient and urine of a non-cancer patient urine of a determination target is, and the determination institute may be a hospital or a research institution.

### Configuration Example of Data Acquisition Device

The data acquisition device 30 in FIG. 6 includes an odor sensor 31, a storage unit 32, and a communication unit 33. The odor sensor 31 forms a urine odor data unit by detecting odor from urine of a subject and outputs the formed urine odor data unit to the storage unit 32.

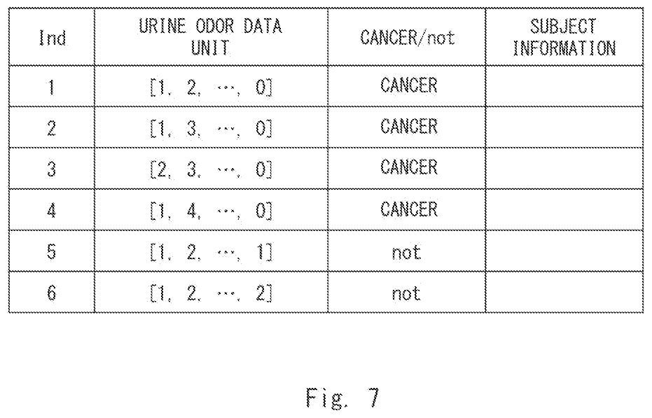

The storage unit 32 stores a urine odor data unit received from the odor sensor 31 in a form of a table (may be hereinafter referred to as a “collected data table”). FIG. 7 is a diagram illustrating an example of a collected data table according to the fifth example embodiment. Each entry in the collected data table illustrated in FIG. 7 includes an index, a urine odor data unit, a cancer label (CANCER/not), and “subject information” as items. For example, the “subject information” may include “sex,” a “height,” a “weight,” a “comorbidity other than cancer,” and a “medication type” about a subject, and a collection condition at the collection of the urine (such as an inpatient or an outpatient) and a collection date. In other words, the “subject information” includes information of the aforementioned “characteristic parameters.” While the collected data table is illustrated in a form of a single table in the example in FIG. 7, the collected data table may be formed as a set of a plurality of tables. For example, the collected data table may be a table set including a first table associating a urine sample ID with a subject ID, a second table associating a urine sample ID with a urine odor data unit, a third table associating a subject ID with subject information, and a fourth table associating a urine sample ID with a cancer label.

The communication unit 33 transmits a collected data table stored in the storage unit 32 to the learning device 40.

### Configuration Example of Learning Device

The learning device 40 in FIG. 6 includes a communication unit 41, a storage unit 42, a selection unit 43, and a determination model formation unit 44.

The communication unit 41 receives a collected data table transmitted from the data acquisition device 30 and outputs the collected data table to the storage unit 42.

The storage unit 42 stores a collected data table received from the communication unit 41.

The selection unit 43 extracts and acquires a learning candidate data unit from each entry in a collected data table stored in the storage unit 42. Specifically, since each entry in the collected data table also includes an item not required for selection processing in the selection unit 43, information about a required item is extracted from each entry and is acquired as a learning candidate data unit.

Then, the selection unit 43 selects, from a plurality of acquired learning candidate data units, part of the plurality of learning candidate data units as a “learning target data set,” based on a “selection rule,” similarly to the selection unit 11 according to any one of the first to fourth example embodiments.

The determination model formation unit 44 forms the aforementioned “determination model” by using a learning target data set selected by the selection unit 43, similarly to the determination model formation units 12 according to the first to fourth example embodiments.

### Configuration Example of Determination Device

The determination device 50 in FIG. 6 includes an odor sensor 51 and a determination unit 52.

The odor sensor 51 forms a determination target urine odor data unit by detecting odor from urine of a subject being a determination target and outputs the formed determination target urine odor data unit to the determination unit 52.

The determination unit 52 determines which of urine of a cancer patient and urine of a non-cancer patient a determination target urine odor data unit received from the odor sensor 51 is related to, by using a determination model formed by the learning device 40. When a characteristic parameter is not used and a urine odor data unit is used in learning of a determination model in the learning device 40, the determination unit 52 makes a determination by using a determination target urine odor data unit received from the odor sensor 51. On the other hand, when a characteristic parameter is used with a urine odor data unit in learning of a determination model in the learning device 40, a value of a characteristic parameter related to a subject being a determination target is also input to the determination unit 52. Then, the determination unit 52 determines which of urine of a cancer patient and urine of a non-cancer patient the determination target urine odor data unit is related to, based on the input determination target urine odor data unit, the input characteristic parameter value, and the determination model.

While the determination device 50 has been described above as a device independent of the data acquisition device 30 and the learning device 40, the determination device 50 is not limited to the above. For example, the determination device 50 may be included in the data acquisition device 30. In this case, the odor sensor 31 and the odor sensor 51 may form a single odor sensor. Further, for example, the determination unit 52 in the determination device 50 may be provided in the learning device 40. In this case, a determination target urine odor data unit formed in the odor sensor 51 may be transmitted to the learning device 40 through a communication unit (unillustrated) in the determination device 50, and the determination unit 52 provided in the learning device 40 may determine which of urine of a cancer patient and urine of a non-cancer patient the determination target urine odor data unit is related to.

Example embodiments according to which the selection unit in the learning device selects, from a plurality of learning candidate data units, part of the plurality of learning candidate data units as a “learning target data set,” based on a “selection rule,” have been described in the aforementioned first to fifth example embodiments. Example embodiments according to which a learning target data set is formed in a learning device by assigning a weight of a loss function used for forming a determination model to each of a plurality of learning candidate data units, based on a balancing rule, will be described in a sixth example embodiment and beyond.

### Sixth Example Embodiment

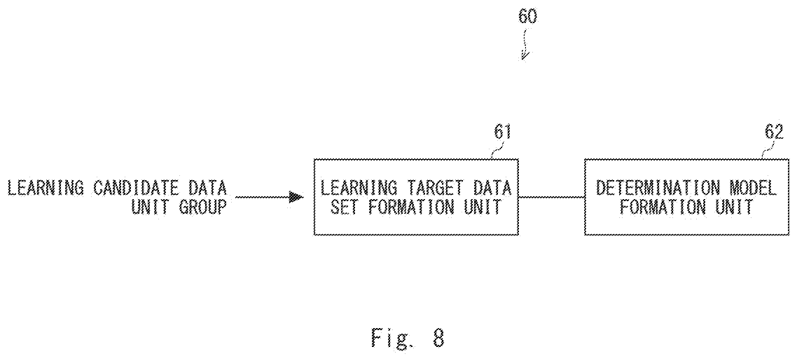

FIG. 8 is a block diagram illustrating an example of a learning device according to a sixth example embodiment. The learning device 60 illustrated in FIG. 8 is a device for learning a “determination model” for determining which of urine of a cancer patient and urine of a non-cancer patient a determination target urine odor data unit is related to, similarly to the learning devices according to the first to fifth example embodiments. The learning device 60 in FIG. 8 includes a learning target data set formation unit 61 and a determination model formation unit 62.

The learning target data set formation unit 61 receives (inputs) a plurality of learning candidate data units (a learning candidate data unit group), similarly to the selection units in the learning devices according to the first to fifth example embodiments.

Then, the learning target data set formation unit 61 forms a “learning target data set” by assigning a “weight” to each of the plurality of learning candidate data units, based on a “balancing rule.” The weight is a weight of a loss function used for forming a determination model. When zero is assigned to a learning candidate data unit as a weight, the learning candidate data unit does not contribute to learning by the determination model formation unit 62. Accordingly, assigning a zero value weight to a learning candidate data unit is equivalent to being excluded from a learning target data set in the “selection processing” in the first example embodiment to the fifth example embodiment.

Returning to the description of FIG. 8, the determination model formation unit 62 forms the aforementioned determination model, based on a learning target data set formed by the learning target data set formation unit 61.

Specifically, the determination model formation unit 62 forms a determination model f in such a way as to minimize the sum total summarizing, for every learning candidate data unit, a value acquired by multiplying a weight w by a value of a loss function loss acquired from a urine odor data unit, a cancer label, and the determination model f in each learning candidate data unit in a learning target data set [see Eqn. (1) below]. The loss function is not particularly limited and, for example, may be cross entropy, hinge loss, exponential loss, or 0-1 loss.

[Math. 1]

argmin_{f}=Σᵢ^{N}wᵢloss(f(xᵢ),yᵢ) (1)

In In Eqn. (1), N denotes the number of learning candidate data units included in a learning target data set. Further, i denotes an i-th learning candidate data unit. Further, wᵢ denotes a weight of an i-th learning candidate data unit. Further, xᵢ denotes an explanatory variable of an i-th learning candidate data unit and at least includes a urine odor data unit of the i-th learning candidate data unit. Further, yᵢ denotes a cancer label.

As described above, the learning target data set formation unit 61 in the learning device 60 forms a learning target data set by assigning a weight of a loss function used for forming a determination model to each of a plurality of input learning candidate data units, based on a balancing rule, according to the sixth example embodiment. The determination model formation unit 62 forms the aforementioned determination model, based on the learning target data set formed by the learning target data set formation unit 61.

With the configuration of the learning device 60, a degree of contribution of each learning candidate data unit to learning by the determination model formation unit 62 can be adjusted. Thus, improved precision of a determination model can be achieved.

### Seventh Example Embodiment

A seventh example embodiment relates to a specific example of the aforementioned “balancing rule.” A basic configuration of a learning device according to the seventh example embodiment is the same as that of the learning device 60 according to the sixth example embodiment and therefore will be described with reference to FIG. 8.

A learning target data set formation unit 61 in the learning device 60 according to the seventh example embodiment forms a “learning target data set” by assigning a “weight” to each of a plurality of input learning candidate data units, based on a “balancing rule,” similarly to the sixth example embodiment.

The “balancing rule” according to the seventh example embodiment includes a sub-rule A1 for balancing, in a “learning target data set,” the sum total of weights assigned to learning candidate data units having a cancer label indicating a cancer patient with the sum total of weights assigned to learning candidate data units having a cancer label indicating a non-cancer patient.

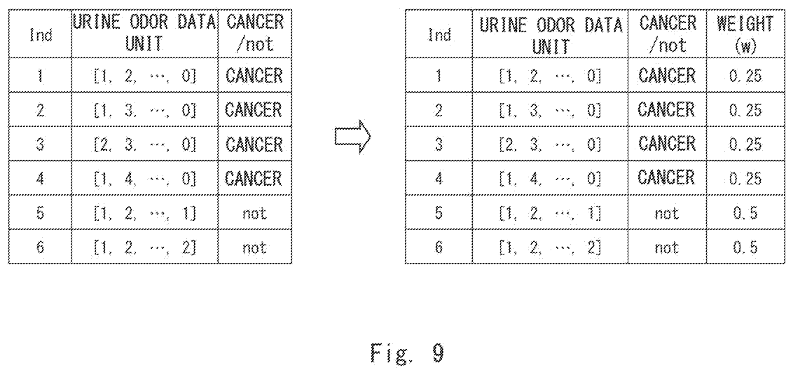

FIG. 9 is a diagram for illustrating an example of a formation method of a learning target data set according to the sixth example embodiment. A left-hand diagram in FIG. 9 illustrates an example of a learning candidate data unit group input to the learning target data set formation unit 61, and a right-hand diagram in FIG. 9 illustrates an example of a “learning target data set” selected by the learning target data set formation unit 61.

Each entry in the left-hand diagram in FIG. 9 is related to a learning candidate data unit and includes an index (Ind), a urine odor data unit, and a cancer label (CANCER/not) as items. Then, as illustrated in the right-hand diagram in FIG. 9, a weight w is assigned to each entry by the learning target data set formation unit 61 in accordance with the “balancing rule.” In the example illustrated in FIG. 9, a weight is assigned to each entry in such a way that the sum total of weights of entries having a cancer label indicating a cancer patient is equal to the sum total of weights of entries having a cancer label indicating a non-cancer patient. Further, in the example illustrated in FIG. 9, a weight of a learning candidate data unit having a cancer label indicating a cancer patient is less than a weight of a learning candidate data unit having a cancer label indicating a non-cancer patient. Therefore, a degree of contribution of a learning candidate data unit having a cancer label indicating a cancer patient to learning by the determination model formation unit 62 is lower compared with a learning candidate data unit having a cancer label indicating a non-cancer patient. While weights assigned to a plurality of learning candidate data units having a cancer label indicating a cancer patient are equal in the example in FIG. 9, the assignment method is not limited to the above and may be different. The same applies to a plurality of learning candidate data units having a cancer label indicating a non-cancer patient.

For example, the determination model formation unit 62 according to the seventh example embodiment forms a determination model in such as way as to minimize a value acquired by Eqn. (1) described above, similarly to the sixth example embodiment. In the example in FIG. 9, xᵢ in Eqn. (1) denotes a urine odor data unit in an i-th learning candidate data unit.

As described above, the learning target data set formation unit 61 in the learning device 60 forms a learning target data set by assigning a weight of a loss function used for forming a determination model to each of a plurality of input learning candidate data units, based on a “balancing rule,” according to the seventh example embodiment. The “balancing rule” includes a sub-rule for balancing, in a “learning target data set,” the sum total of weights assigned to learning candidate data units having a cancer label indicating a cancer patient with the sum total of weights assigned to learning candidate data units having a cancer label indicating a non-cancer patient.

With the configuration of the learning device 60, a degree of contribution of the entire learning candidate data units having a cancer label indicating a cancer patient to learning by the determination model formation unit 62 can be balanced in a “learning target data set” with a degree of contribution of the entire learning candidate data units having a cancer label indicating a non-cancer patient. Thus, improved precision of a determination model can be achieved.

### Eighth Example Embodiment

An eighth example embodiment relates to a variation of the aforementioned “balancing rule.” A basic configuration of a learning device according to the eighth example embodiment is the same as that of the learning device 60 according to the sixth example embodiment and therefore will be described with reference to FIG. 8.

Each learning candidate data unit according to the eighth example embodiment includes a “characteristic parameter” related to a subject in addition to the aforementioned “urine odor data unit” and the aforementioned “cancer label.” The “characteristic parameter” may take N (where N is a natural number equal to or greater than 2) pieces of k-th values (where k=1, . . . , N). In other words, the “characteristic parameter” may take at least a first value and a second value. For example, the “characteristic parameter” may be any one item out of “sex,” a “height,” a “weight,” a “comorbidity other than cancer,” and a “medication type” about a subject, or any combination of the above items.

A learning target data set formation unit 61 in the learning device 60 according to the eighth example embodiment forms a “learning target data set” by assigning a “weight” to each of a plurality of input learning candidate data units, based on a “balancing rule,” similarly to the sixth example embodiment and the seventh example embodiment.

The “balancing rule” according to the eighth example embodiment includes a sub-rule A2 for balancing, in a “learning target data set,” the sum totals of weights of learning candidate data units having k-th values. Specifically, the sub-rule A2 is a rule for balancing, in a learning target data set, the sum total of weights of learning candidate data units having the aforementioned first value with the sum total of weights of learning candidate data units having the aforementioned second value. The sub-rule A2 may be used with the aforementioned sub-rule A1 or may be used singly.

For example, a medication type given to a subject for treatment of a comorbidity other than cancer may be used as the aforementioned characteristic parameter. In this case, the “balancing rule” may include a sub-rule for balancing, in a learning target data set, the sum total of weights of learning candidate data units having a medication type indicating medication affecting urine of a subject and a cancer label indicating a cancer patient with the sum total of weights of learning candidate data units having a medication type indicating medication affecting urine of a subject and a cancer label indicating a non-cancer patient.

As described above, the learning target data set formation unit 61 in the learning device 60 forms a learning target data set by assigning a weight of a loss function used for forming a determination model to each of a plurality of input learning candidate data units, based on a “balancing rule,” according to the eighth example embodiment. Each learning candidate data unit further includes a “characteristic parameter” that is related to a subject and may take at least a first value and a second value. The “balancing rule” includes a sub-rule for balancing, in a learning target data set, the sum total of weights of learning candidate data units having the aforementioned first value with the sum total of weights of learning candidate data units having the aforementioned second value.

With the configuration of the learning device 60, the sum totals of weights between characteristic parameter values can be balanced in a learning target data set. Thus, improved precision of a determination model can be achieved.

### Ninth Example Embodiment

A ninth example embodiment relates to a learning device that can accept specification of a sub-rule to be used out of a plurality of sub-rules different from one another included in a balancing rule.

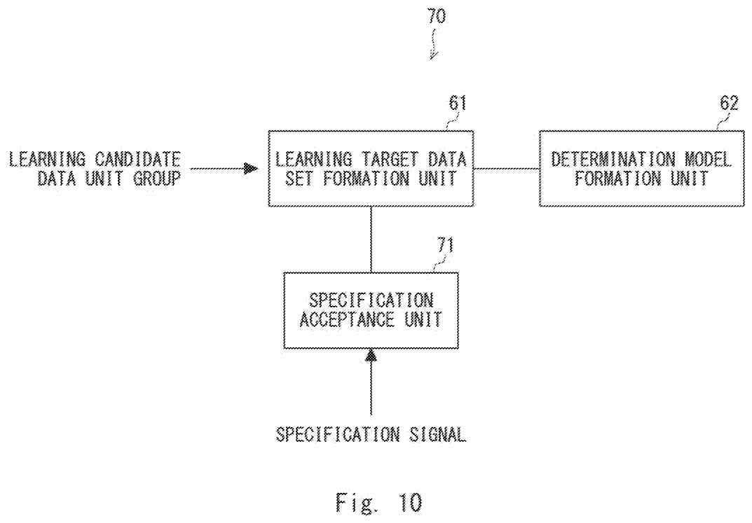

FIG. 10 is a block diagram illustrating an example of a learning device according to the ninth example embodiment. The learning device 70 in FIG. 10 includes a learning target data set formation unit 61, a determination model formation unit 62, and a specification acceptance unit 71.

A “balancing rule” according to the ninth example embodiment includes a plurality of sub-rules different from one another. The specification acceptance unit 71 accepts a “specification signal” indicating a single sub-rule or a combination of a plurality of sub-rules specified by a user operating an operation unit (unillustrated). Then, the specification acceptance unit 71 sets the single sub-rule or the combination of a plurality of sub-rules indicated by the specification signal to the learning target data set formation unit 61 as a “balancing rule to be used.” Thus, the learning target data set formation unit 61 can form a learning target data set by assigning a weight of a loss function used for forming a determination model to each input learning candidate data unit, based on the “balancing rule to be used” set by the specification acceptance unit 71.

As described above, the specification acceptance unit 71 in the learning device 70 accepts a “specification signal” indicating a single sub-rule or a combination of a plurality of sub-rules specified by a user operating the operation unit (unillustrated), according to the ninth example embodiment. Then, the specification acceptance unit 71 sets the single sub-rule or the combination of a plurality of sub-rules indicated by the specification signal to the learning target data set formation unit 61 as a “balancing rule to be used.”

With the configuration of the learning device 70, a learning target data set” can be formed by using a balancing rule matching user needs.

### Tenth Example Embodiment

A tenth example embodiment is related to a cancer examination system including a learning device.

### Outline of Cancer Examination System

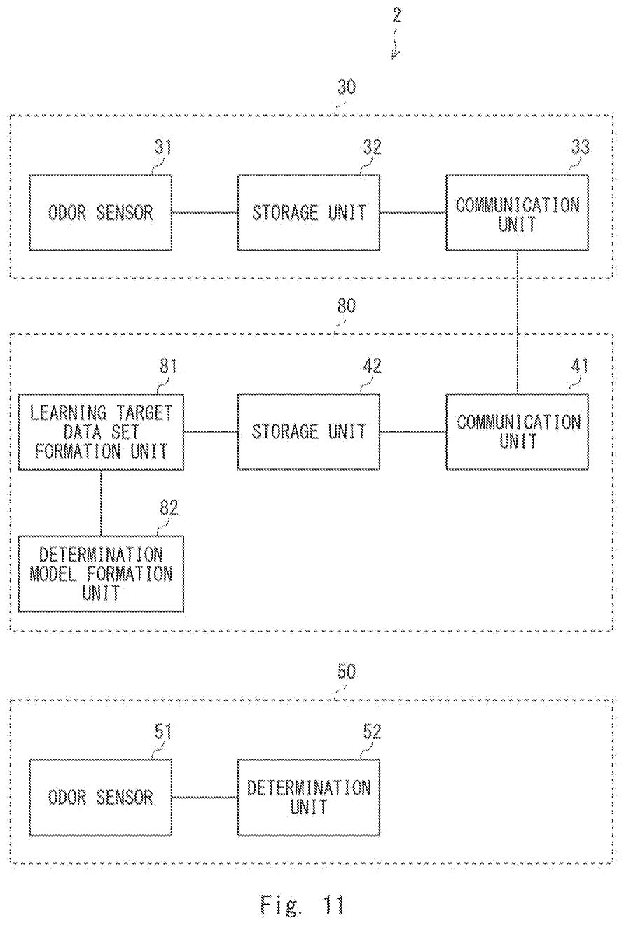

FIG. 11 is a block diagram illustrating an example of a cancer diagnostic system according to the tenth example embodiment. The cancer diagnostic system 2 in FIG. 10 includes a data acquisition device 30, a learning device 80, and a determination device 50. For example, the learning device 80 may be installed in a hospital or a research institution or may be constructed on a cloud. The data acquisition device 30 and the determination device 50 are the same as those according to the fifth example embodiment.

### Configuration Example of Learning Device

The learning device 80 in FIG. 11 includes a communication unit 41, a storage unit 42, a learning target data set formation unit 81, and a determination model formation unit 82.

The learning target data set formation unit 81 extracts and acquires a learning candidate data unit from each entry in a collected data table stored in the storage unit 42. Specifically, since each entry in the collected data table also includes an item not required for selection processing in the selection unit 43, information about a required item is extracted from each entry and is acquired as a learning candidate data unit.

Then, the learning target data set formation unit 81 forms a “learning target data set” by assigning a “weight” to each of a plurality of learning candidate data units, based on a “balancing rule,” similarly to the learning target data set formation unit 61 according to any one of the sixth to ninth example embodiments.

The determination model formation unit 82 forms the aforementioned “determination model” by using a learning target data set formed by the learning target data set formation unit 81, similarly to the determination model formation units 62 according to the sixth to ninth example embodiments.

### Other Example Embodiments

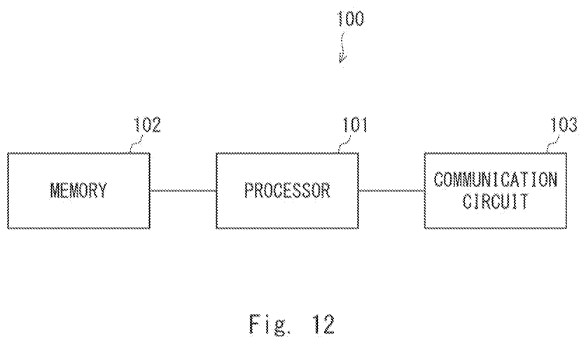

FIG. 12 is a diagram illustrating a hardware configuration example of a learning device. The learning device 100 in FIG. 12 includes a processor 101, a memory 102, and a communication circuit 103. For example, the processor 101 may be a microprocessor, a micro processing unit (MPU), or a central processing unit (CPU). The processor 101 may include a plurality of processors. The memory 102 is configured with a combination of a volatile memory and a nonvolatile memory. The memory 102 may include a storage placed apart from the processor 101. In this case, the processor 101 may access the memory 102 through an unillustrated I/O interface.

Each of the learning devices 10, 20, 40, 60, 70, and 80 according to the first to tenth example embodiments may include the hardware configuration illustrated in FIG. 12. The selection units 11 and 43, the determination model formation units 12 and 44, the specification acceptance unit 21, the learning target data set formation units 61 and 81, the determination model formation units 62 and 82, and the specification acceptance unit 71 in the learning devices 10, 20, 40, 60, 70, and 80 according to the first to tenth example embodiments may be provided by the processor 101 reading and executing a program stored in the memory 102. Further, the storage unit 42 may be provided by the memory 102. Further, the communication unit 41 may be provided by the communication circuit 103. The program is stored by using various types of non-transitory computer-readable media and can be supplied to the learning devices 10, 20, 40, 60, 70, and 80. Examples of the non-transitory computer-readable medium include magnetic recording media (such as a flexible disk, a magnetic tape, and a hard disk drive), magneto-optical recording media (such as a magneto-optical disk). Examples of the non-transitory computer-readable medium further include a CD-read only memory (ROM), a CD-R, and a CD-R/W. Furthermore, examples of the non-transitory computer-readable medium include semiconductor memories. Examples of the semiconductor memory include a mask ROM, a programmable ROM (PROM), an erasable PROM (EPROM), a flash ROM, and a random access memory (RAM). Further, the program may be supplied to the learning devices 10, 20, 40, 60, 70, and 80 by various types of transitory computer-readable media. Examples of the transitory computer-readable medium include an electric signal, an optical signal, and an electromagnetic wave. The transitory computer-readable medium can supply the program to the learning devices 10, 20, 40, 60, 70, and 80 through a wired communication channel such as an electric cable or an optical fiber, or a wireless communication channel.

While the present invention has been described above with reference to the example embodiments, the present invention is not limited to the above. Various changes and modifications that may be understood by a person skilled in the art may be made to the configurations and details of the present invention without departing from the spirit and scope of the present invention.

The whole or part of the example embodiments disclosed above can be described as, but not limited to, the following supplementary notes.

### Supplementary Note A1

A learning device including:

a selection unit configured to, from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, select part of the plurality of learning candidate data units as a learning target data set, based on a selection rule; and

a determination model formation unit configured to form a determination model for determining which of urine of a cancer patient and urine of a non-cancer patient a determination target urine odor data unit is related to, by using the selected learning target data set.

### Supplementary Note A2

The learning device according to Supplementary Note A1, wherein

each learning candidate data unit further includes a characteristic parameter that is related to the subject and may take at least a first value and a second value, and

the selection rule includes a first sub-rule for balancing, in the learning target data set, the number of the learning candidate data unit having the first value with the number of the learning candidate data unit having the second value.

### Supplementary Note A3

The learning device according to Supplementary Note A2, wherein the selection rule further includes a second sub-rule for balancing, in the learning target data set, the number of the learning candidate data unit having the cancer label indicating a cancer patient with the number of the learning candidate data unit having the cancer label indicating a non-cancer patient.

### Supplementary Note A4

The learning device according to Supplementary Note A2 or A3, wherein the characteristic parameter is any one item out of sex, a height, a weight, a comorbidity other than cancer, and a medication type about the subject, or any combination of the above items.

### Supplementary Note A5

The learning device according to any one of Supplementary Notes A2 to A4, wherein

the selection rule includes a plurality of sub-rules different from one another, and

the learning device further includes a specification acceptance unit configured to accept specification of a sub-rule used for selection of the learning target data set by the selection unit out of the plurality of sub-rules.

### Supplementary Note A6

The learning device according to any one of Supplementary Notes A2 to A5, wherein the determination model formation unit forms the determination model by using the urine odor data unit and a cancer label without using, in learning, the characteristic parameter included in each learning candidate data unit in the selected learning target data set.

### Supplementary Note A7

The learning device according to Supplementary Note A1, wherein

each learning candidate data unit further includes a medication type given to the subject for treatment of a comorbidity other than cancer, and

the selection rule includes a third sub-rule for balancing, in the learning target data set, the number of the learning candidate data unit having the medication type indicating medication affecting urine of the subject and the cancer label indicating a cancer patient with the number of the learning candidate data unit having the medication type indicating medication affecting urine of the subject and the cancer label indicating a non-cancer patient.

### Supplementary Note A8

The learning device according to any one of Supplementary Notes A1 to A7, wherein the cancer label further includes at least one item out of a type of cancer of the subject and progress of cancer of the subject.

### Supplementary Note A9

A learning method including:

from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, selecting part of the plurality of learning candidate data units as a learning target data set, based on a selection rule; and

forming a determination model for determining which of urine of a cancer patient and urine of a non-cancer patient a determination target urine odor data unit is related to, by using the selected learning target data set.

### Supplementary Note A10

A control program for causing a learning device to execute processing of:

from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, selecting part of the plurality of learning candidate data units as a learning target data set, based on a selection rule; and

forming a determination model for determining which of urine of a cancer patient and urine of a non-cancer patient a determination target urine odor data unit is related to, by using the selected learning target data set.

### Supplementary Note B1

A learning device including:

a learning target data set formation unit configured to form a learning target data set by assigning, based on a balancing rule, a weight of a loss function used for forming a determination model to each of a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient; and

a determination model formation unit configured to, based on the formed learning target data set, form the determination model for determining which of urine of a cancer patient and urine of a non-cancer patient a determination target urine odor data unit is related to.

### Supplementary Note B2

The learning device according to Supplementary Note B1, wherein the balancing rule includes a sub-rule for balancing, in the learning target data set, a sum total of a weight assigned to the learning candidate data unit having a cancer label indicating that the subject is a cancer patient with a sum total of a weight assigned to the learning candidate data unit having a cancer label indicating that the subject is a non-cancer patient.

### Supplementary Note B3

The learning device according to Supplementary Note B1, wherein

each learning candidate data unit further includes a characteristic parameter that is related to the subject and may take at least a first value and a second value, and

the balancing rule includes a sub-rule for balancing, in the learning target data set, a sum total of a weight assigned to the learning candidate data unit having the first value with a sum total of a weight assigned to the learning candidate data unit having the second value.

### Supplementary Note B4

The learning device according to Supplementary Note B3, wherein the characteristic parameter is any one item out of sex, a height, a weight, a comorbidity other than cancer, and a medication type about the subject, or any combination of the above items.

### Supplementary Note B5

The learning device according to Supplementary Note B3 or B4, wherein

the balancing rule includes a plurality of sub-rules different from one another, and

the learning device further includes a specification acceptance unit configured to accept specification of a sub-rule used for formation of the learning target data set by the learning target data set formation unit out of the plurality of sub-rules.

### Supplementary Note B6

The learning device according to Supplementary Note B1, wherein

each learning candidate data unit further includes a medication type given to the subject for treatment of a comorbidity other than cancer, and

the balancing rule includes a sub-rule for balancing, in the learning target data set, a sum total of a weight of the learning candidate data unit having the medication type indicating medication affecting urine of the subject and the cancer label indicating a cancer patient with a sum total of a weight of the learning candidate data unit having the medication type indicating medication affecting urine of the subject and the cancer label indicating a non-cancer patient.

### Supplementary Note B7

The learning device according to any one of Supplementary Notes B1 to B6, wherein the learning target data set formation unit excludes part of the plurality of learning candidate data units from the learning target data set by assigning the weight with a zero value to the part of the learning candidate data units.

### Supplementary Note B8

The learning device according to any one of Supplementary Notes B1 to B7, wherein the cancer label further includes at least one item out of a type of cancer of the subject and progress of cancer of the subject.

### Supplementary Note B9

A learning method including:

forming a learning target data set by assigning, based on a balancing rule, a weight of a loss function used for forming a determination model to each of a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient; and,

based on the formed learning target data set, forming the determination model for determining which of urine of a cancer patient and urine of a non-cancer patient a determination target urine odor data unit is related to.

### Supplementary Note B10

A control program for causing a learning device to execute processing of:

forming a learning target data set by assigning, based on a balancing rule, a weight of a loss function used for forming a determination model to each of a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient; and,

based on the formed learning target data set, forming the determination model for determining which of urine of a cancer patient and urine of a non-cancer patient a determination target urine odor data unit is related to.

This application is based upon and claims the benefit of priority from Japanese patent application No. 2019-074032, filed on Apr. 9, 2019, the disclosure of which is incorporated herein in its entirety by reference.

### REFERENCE SIGNS LIST

1 CANCER DIAGNOSTIC SYSTEM
2 CANCER DIAGNOSTIC SYSTEM
10 LEARNING DEVICE
11 SELECTION UNIT
12 DETERMINATION MODEL FORMATION UNIT
20 LEARNING DEVICE
21 SPECIFICATION ACCEPTANCE UNIT
30 DATA ACQUISITION DEVICE
31 ODOR SENSOR
32 STORAGE UNIT
33 COMMUNICATION UNIT
40 LEARNING DEVICE
41 COMMUNICATION UNIT
42 STORAGE UNIT
43 SELECTION UNIT
44 DETERMINATION MODEL FORMATION UNIT
50 DETERMINATION DEVICE
51 ODOR SENSOR
52 DETERMINATION UNIT
60 LEARNING DEVICE
61 LEARNING TARGET DATA SET FORMATION UNIT
62 DETERMINATION MODEL FORMATION UNIT
70 LEARNING DEVICE
71 SPECIFICATION ACCEPTANCE UNIT
80 LEARNING DEVICE
81 LEARNING TARGET DATA SET FORMATION UNIT
82 DETERMINATION MODEL FORMATION UNIT

1. A learning device comprising:
hardware including at least one processor and at least one memory;
selection for unit implemented at least by the hardware and that selects, from a plurality of learning candidate data units, part of the plurality of learning candidate data units as a learning target data set, based on a selection rule, wherein the plurality of learning candidate data units respectively are related to a plurality of subjects including a plurality of cancer patients and a plurality of non-cancer patients, and wherein each learning candidate data unit includes a urine odor data unit acquired from urine of a related subject and a cancer label indicating whether the related subject is a cancer patient or a non-cancer patient; and
determination model formation unit implemented at least by the hardware and that forms, by using the selected learning target data set, a determination model for determining which of urine of a cancer patient and urine of a non-cancer patient a determination target urine odor data unit is related to.

2. The learning device according to claim 1, wherein
each learning candidate data unit further includes a characteristic parameter that is related to the subject and may take at least a first value and a second value, and
the selection rule includes a first sub-rule for balancing, in the learning target data set, the number of the learning candidate data unit having the first value with the number of the learning candidate data unit having the second value.

3. The learning device according to claim 2, wherein the selection rule further includes a second sub-rule for balancing, in the learning target data set, the number of the learning candidate data unit having the cancer label indicating a cancer patient with the number of the learning candidate data unit having the cancer label indicating a non-cancer patient.

4. The learning device according to claim 2, wherein the characteristic parameter is any one item out of sex, a height, a weight, a comorbidity other than cancer, and a medication type about the subject, or any combination of the above items.

5. The learning device according to claim 2, wherein
the selection rule includes a plurality of sub-rules different from one another, and
the learning device further comprises specification acceptance unit implemented at least by the hardware and that accepts specification of a sub-rule used for selection of the learning target data set by the selection unit out of the plurality of sub-rules.

6. The learning device according to claim 2, wherein the determination model formation unit forms the determination model by using the urine odor data unit and a cancer label without using, in learning, the characteristic parameter included in each learning candidate data unit in the selected learning target data set.

7. The learning device according to claim 1, wherein
each learning candidate data unit further includes a medication type given to the subject for treatment of a comorbidity other than cancer, and
the selection rule includes a third sub-rule for balancing, in the learning target data set, the number of the learning candidate data unit having the medication type indicating medication affecting urine of the subject and the cancer label indicating a cancer patient with the number of the learning candidate data unit having the medication type indicating medication affecting urine of the subject and the cancer label indicating a non-cancer patient.

8. The learning device according to claim 1, wherein the cancer label further includes at least one item out of a type of cancer of the subject and progress of cancer of the subject.

9. A learning method comprising:
from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, selecting part of the plurality of learning candidate data units as a learning target data set, based on a selection rule; and
forming a determination model for determining which of urine of a cancer patient and urine of a non-cancer patient a determination target urine odor data unit is related to, by using the selected learning target data set.

10. A non-transitory computer-readable medium storing a control program for causing a learning device to execute processing of:
from a plurality of learning candidate data units respectively related to a plurality of subjects including a plurality of cancer patients and a plurality of non-cancer patients, each learning candidate data unit at least including a urine odor data unit acquired from urine of a related subject and a cancer label at least indicating whether the related subject is a cancer patient or a non-cancer patient, selecting part of the plurality of learning candidate data units as a learning target data set, based on a selection rule; and
forming a determination model for determining which of urine of a cancer patient and urine of a non-cancer patient a determination target urine odor data unit is related to, by using the selected learning target data set.

11.-20. (canceled)
